(19) United States
(12) Patent Application Publication
(10) Pub. No.: US 2025/0136560 A1
(43) Pub. Date: May 1, 2025

(54) PROCESS TO PRODUCE HEXAHYDRO-1,3,5-TRINITRO-1,3,5-TRIAZINE AND OCTAHYDRO-1,3,5,7-TETRANITRO-1,3,5,7-TETRAZOCINE WITH DIRECT RECYCLE OF SPENT ACID

(71) Applicant: James E. PHILLIPS, Kingsport, TN (US)

(72) Inventor: James E. PHILLIPS, Kingsport, TN (US)

(21) Appl. No.: 18/865,952

(22) PCT Filed: May 12, 2023
(86) PCT No.: PCT/US2023/022020

§ 371 Date: Nov. 14, 2024

Related U.S. Application Data
(60) Provisional application No. 63/345,361, filed on May 24, 2022.
(60) Provisional application No. 63/430,220, filed on Dec. 5, 2022.

(51) Int. Cl. C07D 251/06; C07D 257/02

(52) U.S. Cl. CPC C07D 251/06; C07D 257/02

(57) ABSTRACT

During the production of Hexahydro-1,3,5-trinitro-1,3,5-triazine and Octahydro-1,3,5,7-tetranitro-1,3,5,7-tetrazocine via the Bachmann nitrolysis process, it is necessary to recover the entire mass of acetic acid and restore it to an anhydrous state via azeotropic distillation. The azeotropic distillation process is resource intensive and is a limiting step with respect to time. Limiting the amount of water in the spent acetic acid allows restoration of an anhydrous state by the addition of acetic anhydride and avoiding azeotropic distillation. The amount of ammonium nitrate in the resulting anhydrous spent acid is accounted for, and the recovered anhydrous spent acid is used directly in successive nitrolysis batches with minimal processing. The yield and quality of the Hexahydro-1,3,5-trinitro-1,3,5-triazine and Octahydro-1,3,5,7-tetranitro-1,3,5,7-tetrazocine thus produced is equal to the yield and quality of the legacy process Hexahydro-1,3,5-trinitro-1,3,5-triazine and Octahydro-1,3,5,7-tetranitro-1,3,5,7-tetrazocine employing aqueous workup conditions.

### CROSS-REFERENCE TO RELATED APPLICATIONS

This application is a national phase entry under 35 U.S.C. § 371 of International Application PCT/US2023/022020, filed May 12, 2023, which claims priority to U.S. provisional patent application No. 63/345,361, filed May 24, 2022 and U.S. provisional patent application No. 63/430,220, filed Dec. 5, 2022, each of which is hereby incorporated by reference in its entirety.

### BACKGROUND

Hexahydro-1,3,5-trinitro-1,3,5-triazine (“RDX”) and Octahydro-1,3,5,7-tetranitro-1,3,5,7-tetrazocine (“HMX”) are high energy explosives produced on industrial scale by the Bachmann process. The Bachmann process consists of nitrolysis of 1,3,5,7-tetraazatricyclo[3.3.1.1^{3,7}]decane via a solution of nitric acid and ammonium nitrate in acetic acid in the presence of a dehydrating agent, acetic anhydride. The process is summarized in FIG. 1—Conventional Hexahydro-1,3,5-trinitro-1,3,5-triazine Flow Diagram and FIG. 3—Conventional Octahydro-1,3,5,7-tetranitro-1,3,5,7-tetrazocine Flow Diagram.

The standard reactor heel for Hexahydro-1,3,5-trinitro-1,3,5-triazine consists of acetic acid, ammonium nitrate, nitric acid and acetic anhydride. The heel for Octahydro-1,3,5,7-tetranitro-1,3,5,7-tetrazocine excludes ammonium nitrate and nitric acid. The molar ratios of acetic acid, ammonium nitrate, nitric acid, and acetic anhydride in the standard reactor heel play an important role in the yield of the final product, and the ratio of Hexahydro-1,3,5-trinitro-1,3,5-triazine to Octahydro-1,3,5,7-tetranitro-1,3,5,7-tetrazocine in the final product. Deviation from the standard reactor heel molar ratios of acetic acid, ammonium nitrate, nitric acid and acetic anhydride will impact the yield and molar ratios of Hexahydro-1,3,5-trinitro-1,3,5-triazine and Octahydro-1,3,5,7-tetranitro-1,3,5,7-tetrazocine in the final product. Introduction of alternate nitrate salts other than ammonium nitrate (i.e. sodium nitrate), in part, or in whole, will impact the yield and molar ratios of Hexahydro-1,3,5-trinitro-1,3,5-triazine and Octahydro-1,3,5,7-tetranitro-1,3,5,7-tetrazocine in the final product. The reagents are added concurrently via three feed streams; 1.) a solution of 1,3,5,7-tetraazatricyclo[3.3.1.1^{3,7}]decane in acetic acid, 2.) a solution of ammonium nitrate in nitric acid, 3.) acetic anhydride. Upon complete addition of the feed streams, the slurry is aged at the prescribed temperature for 45 minutes (Hexahydro-1,3,5-trinitro-1,3,5-triazine: 65° C., Octahydro-1,3,5,7-tetranitro-1,3,5,7-tetrazocine: 44° C.) followed by quenching of the excess acetic anhydride with water to approximately 35 wt % water (20 wt % for Octahydro-1,3,5,7-tetranitro-1,3,5,7-tetrazocine). The aqueous/acetic acid slurry is heated to 98-100° C. for the prescribed time (Hexahydro-1,3,5-trinitro-1,3,5-triazine: 30 min., Octahydro-1,3,5,7-tetranitro-1,3,5,7-tetrazocine: 120 min.), followed by cooling and filtering. The crude solids are dried and recrystallized. The aqueous spent acid is sent to the evaporators to remove the nonvolatile components (Hexahydro-1,3,5-trinitro-1,3,5-triazine, Octahydro-1,3,5,7-tetranitro-1,3,5,7-tetrazocine, ammonium nitrate, remaining linear nitramines). The resulting spent acid stream is then azeotropically distilled to separate the water and methanoic acid from the feed stream to produce glacial acetic acid to complete the process cycle. A portion of the distilled glacial acetic acid is directed to the ketene furnaces to regenerate the dehydrating agent acetic anhydride. The process requires the entire volume of glacial acetic acid be derived from the spent acetic acid recovered from the nitrolysis reactions.

Modeling studies and evidence collected over the past two decades demonstrate the inclusion of methanoic acid as a minor component in a water acetic acid mixture transforms a simple, economically viable distillation step into a more complicated, resource intensive process. If a distillation facility is not specifically designed to accommodate a feed stream of acetic acid and water with approximately 0.25-0.50 wt % methanoic acid, process difficulties are likely to arise. When such process problems have been encountered to date with an inadequate distillation design, the solutions to the problem have resulted in dramatically increased energy usage, reduced glacial acetic acid output and rapid corrosion of the distillation columns requiring increased maintenance and downtimes. An improved process which reduces the volume of spent acid requiring azeotropic distillation to complete the cycle would introduce significant efficiencies and cost savings in producing Hexahydro-1,3,5-trinitro-1,3,5-triazine and Octahydro-1,3,5,7-tetranitro-1,3,5,7-tetrazocine.

Throughout this disclosure, all percentages are in weight percent unless indicated to the contrary.

### PRIOR ART STATEMENT

According to U.S. Pat. No. 4,163,845, “Recycle of Spent Acid in Nitrolysis of 1,3,5,7-tetraazatricyclo[3.3.1.1^{3,7}]decane to Hexahydro-1,3,5-trinitro-1,3,5-triazine” by Brumley et al., West German Pat. No. 1939541 (“Octahydro-1,3,5,7-tetranitro-1,3,5,7-tetrazocine from 1,3,5,7-tetraazatricyclo[3.3.1.1^{3,7}]decane by a Simplified-Nitration Process” by PRB NV) and French Pat. No. 2053804 (“Octahydro-1,3,5,7-tetranitro-1,3,5,7-tetrazocine Production Without Dilution of Reaction Mixture—Reaction Mixture” by France Etat) both relate to the production of Octahydro-1,3,5,7-tetranitro-1,3,5,7-tetrazocine and disclose heating of the reaction mass, after nitrolysis without dissolution with water, to destroy by-products and recycling of the resulting spent acid. However, the French patent distills the spent acid to recover its content of acetic acid, which has been separated from Octahydro-1,3,5,7-tetranitro-1,3,5,7-tetrazocine and Hexahydro-1,3,5-trinitro-1,3,5-triazine, while the German patent recycles the spent acid which apparently still contains excess nitric acid and acetic anhydride. Even though the Octahydro-1,3,5,7-tetranitro-1,3,5,7-tetrazocine nitrolysis and Hexahydro-1,3,5-trinitro-1,3,5-triazine nitrolysis employ the same reagents, these processes are very different, since the proportions of the reagents, reaction temperatures, reaction mechanisms and precursors and by-product nitramines are significantly different.

U.S. Pat. No. 4,163,845, relating to the production of Hexahydro-1,3,5-trinitro-1,3,5-triazine, discloses treating the reaction slurry following the nitrolysis age step with sufficient water to quench the excess acetic anhydride and produce a slurry with 1-2 wt % water content. The resulting slurry is heated to 90-100° C. until the linear nitramines are destroyed. The cooled filtrate is either used directly, or the excess nitric acid is neutralized before proceeding with various experiments. Experiments investigating the recycling of the 1.0 wt % aqueous spent acid to the 1,3,5,7-tetraazatricyclo[3.3.1.1^{3,7}]decane dissolution step show minimal impact on Hexahydro-1,3,5-trinitro-1,3,5-triazine yield. Experiments investigating recycling 1.0 and 2.0 wt % aqueous spent acid which is dehydrated with the appropriate quantity of acetic anhydride prior to the 1,3,5,7-tetraazatricyclo[3.3.1.1^{3,7}]decane dissolution step, show a 23% decrease in Hexahydro-1,3,5-trinitro-1,3,5-triazine yield. Experiments directed toward neutralizing the excess nitric acid with sodium nitrate in the spent acid prior to the dissolution of 1,3,5,7-tetraazatricyclo[3.3.1.1^{3,7}]decane show a 17% decrease in Hexahydro-1,3,5-trinitro-1,3,5-triazine yield. Experiments directed toward neutralizing the excess nitric acid in the spent acid used in both the 1,3,5,7-tetraazatricyclo[3.3.1.1^{3,7}]decane dissolution step and formation of the nitrolysis heel, show a 36% decrease in Hexahydro-1,3,5-trinitro-1,3,5-triazine yield. The solubility of ammonium nitrate in acetic acid increases as the wt % water increases. The solubility of ammonium nitrate in 1% and 2% aqueous spent acid is greater than anhydrous spent acid. In U.S. Pat. No. 4,163,845, the aqueous spent acid, with ammonium nitrate present, is brought forward to the subsequent heel and the 1,3,5,7-tetraazatricyclo[3.3.1.1^{3,7}]decane dissolution step then dehydrated with the appropriate quantity of acetic anhydride. This introduces excess ammonium nitrate to the standard reactor heel. U.S. Pat. No. 4,163,845 does not account for this additional ammonium nitrate introduced to the subsequent recycled reactor heel. In U.S. Pat. No. 4,163,845, the spent acid with nitric acid present is neutralized with sodium acetate. This introduces sodium nitrate to the subsequent reactor heel. A mixture of sodium nitrate and ammonium nitrate in the subsequent recycled reactor heel negatively impacts the yield and molar ratio of Hexahydro-1,3,5-trinitro-1,3,5-triazine and Octahydro-1,3,5,7-tetranitro-1,3,5,7-tetrazocine.

U.S. Pat. No. 4,086,228 “Process for Preparing Octahydro-1,3,5,7-tetranitro-1,3,5,7-tetrazocine” by Solomon, et al related to an improved Octahydro-1,3,5,7-tetranitro-1,3,5,7-tetrazocine process and discloses adding 1,3,5,7-tetraazatricyclo[3.3.1.1^{3,7}]decane to the heel prior to commencing the nitrolysis step in the conventional process. The amount of 1,3,5,7-tetraazatricyclo[3.3.1.1^{3,7}]decane added to the heel prior to commencing the addition of the 3 feedstreams is equal to 20-25% of that which is eventually added via the 1,3,5,7-tetraazatricyclo[3.3.1.1^{3,7}]decane/acetic acid feedstream. The total 1,3,5,7-tetraazatricyclo[3.3.1.1^{3,7}]decane addition equating to 120-125%. This process adjustment succeeds in increasing the overall Octahydro-1,3,5,7-tetranitro-1,3,5,7-tetrazocine yield by 24%. This process adjustment also increases the Hexahydro-1,3,5-trinitro-1,3,5-triazine yield in the reaction to approximately 12% relative to Octahydro-1,3,5,7-tetranitro-1,3,5,7-tetrazocine. The conventional Octahydro-1,3,5,7-tetranitro-1,3,5,7-tetrazocine process produces Hexahydro-1,3,5-trinitro-1,3,5-triazine as a side product which does not typically exceed 2% relative to Octahydro-1,3,5,7-tetranitro-1,3,5,7-tetrazocine because purging the Hexahydro-1,3,5-trinitro-1,3,5-triazine side product above 2% relative is not economically viable. The prior art does not disclose a method for purging the excess Hexahydro-1,3,5-trinitro-1,3,5-triazine from Octahydro-1,3,5,7-tetranitro-1,3,5,7-tetrazocine.

### SUMMARY OF THE INVENTION

The present invention provides a direct recycle of spent acid process for making hexahydro-1,3,5-trinitro-1,3,5-triazine and octahydro-1,3,5,7-tetranitro-1,3,5,7-tetrazocine.

### BRIEF DESCRIPTION OF THE DRAWINGS

FIG. 1—Conventional Process Hexahydro-1,3,5-trinitro-1,3,5-triazine.

FIG. 2—Conventional Hexahydro-1,3,5-trinitro-1,3,5-triazine Process Modified According to the Present Invention.

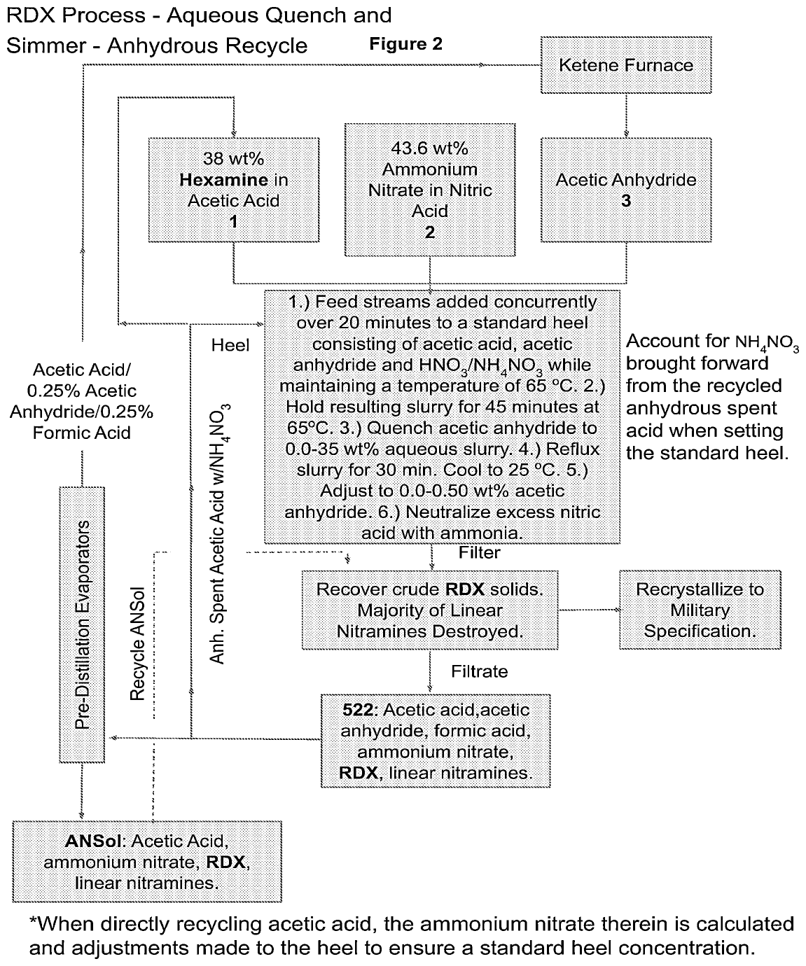

FIG. 3—Conventional Hexahydro-1,3,5-trinitro-1,3,5-triazine Process Modified According to the Present Invention including aqueous quench without simmer and with Direct Recycle.

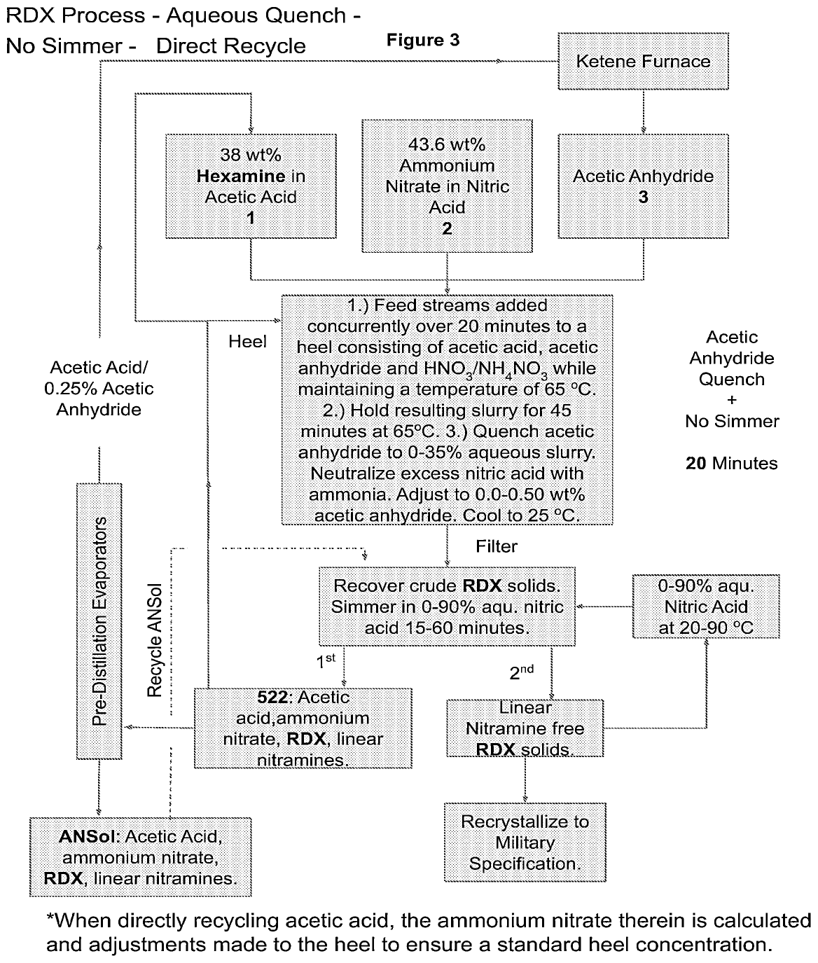

FIG. 4—Conventional Process Octahydro-1,3,5,7-tetranitro-1,3,5,7-tetrazocine.

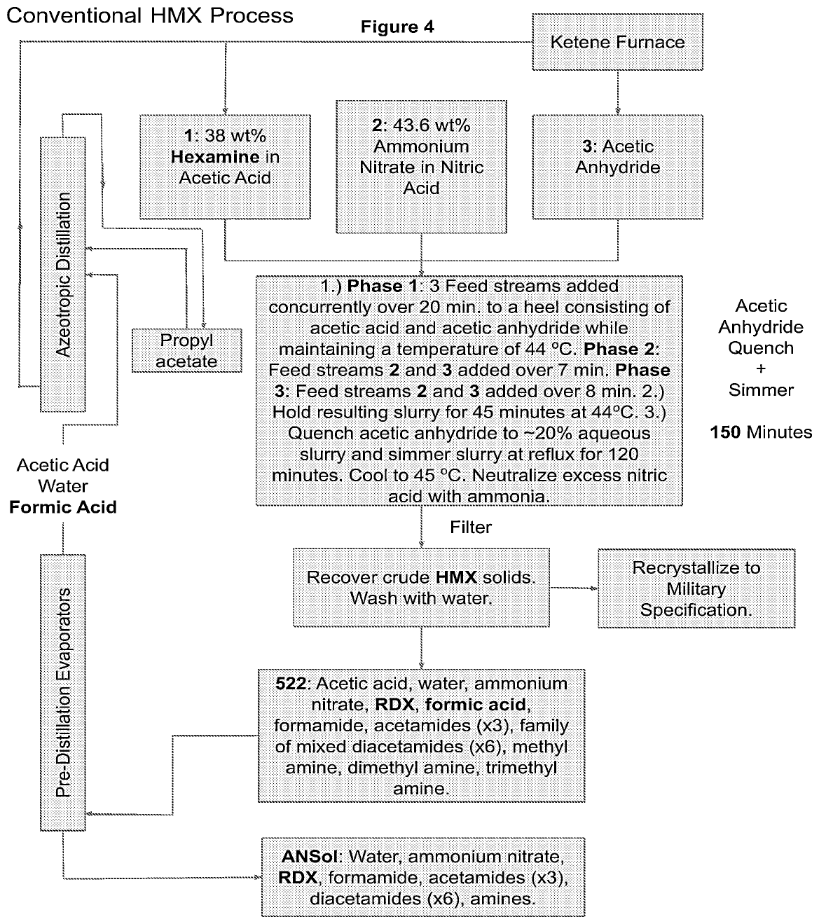

FIG. 5—Conventional Octahydro-1,3,5,7-tetranitro-1,3,5,7-tetrazocine Process Modified According to the Present Invention.

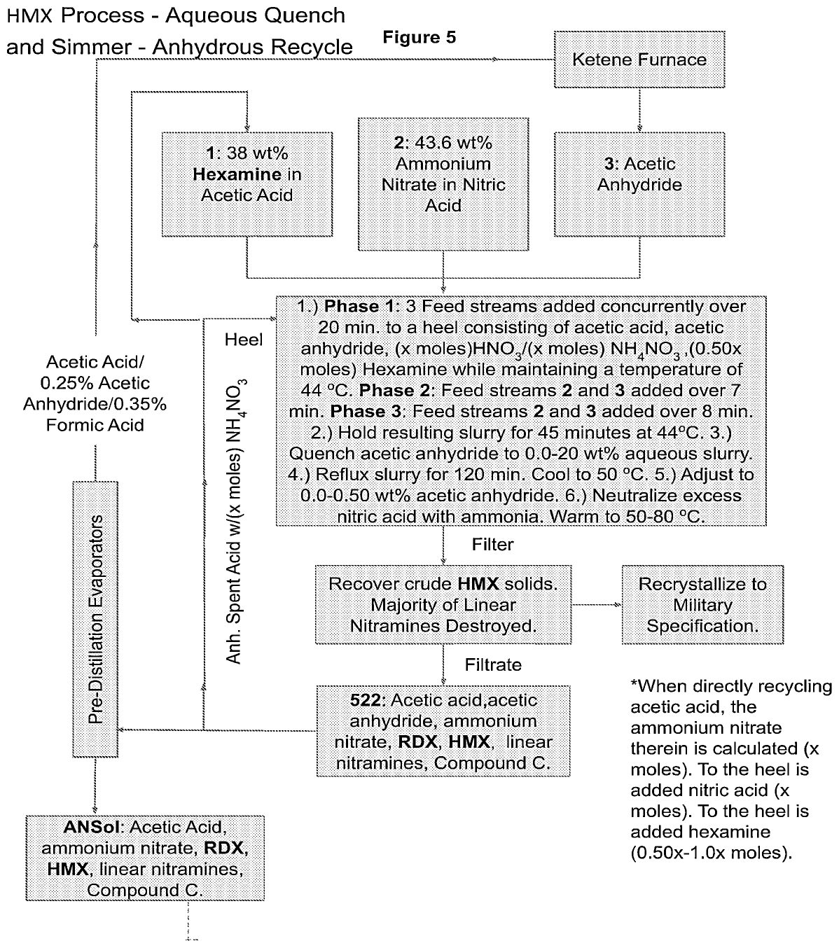

FIG. 6—Conventional Octahydro-1,3,5,7-tetranitro-1,3,5,7-tetrazocine Process Modified According to the Present Invention including aqueous quench without simmer and with Direct Recycle.

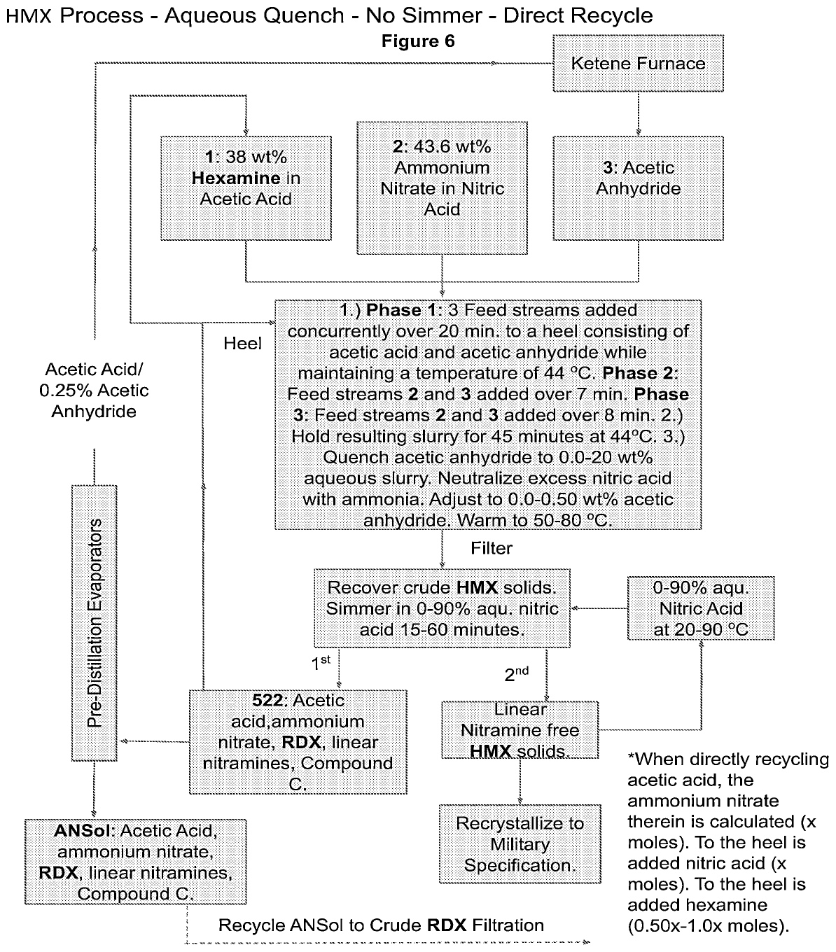

### DETAILED DESCRIPTION

### Direct Recycle of Spent Acid Nitramine Production Process

Throughout this disclosure, all recited temperatures are the central point in a range of +/−5° C. and all recited times are the central point in a range of +/−5 minutes.

In some embodiments, the term “in proportions greater than necessary,” when used in reference to amounts of one or more starting materials or reactants for a chemical reaction or process, refers to stoichiometric amounts of the respective one or more starting materials or reactants. “Proportions greater than necessary” generally means that starting materials or reactants are present in stoichiometric excess, i.e. are present in amounts greater than would be required according to predicted stoichiometry for a given chemical reaction or process. In general, for a given chemical reaction or process, at least one reactant or starting material will be stoichiometrically limiting, and other reactants or starting materials will generally be in excess. For example, excess reactants or starting materials may be present in about 1.1 to about 10 times molar excess, or more. The term “proportions greater than necessary” is not intended to be limiting, and rather a person skilled in the art would appreciate that various reactants or starting materials may be present in excess to perform a given chemical reaction or process.

Conventional Process Hexahydro-1,3,5-trinitro-1,3,5-triazine

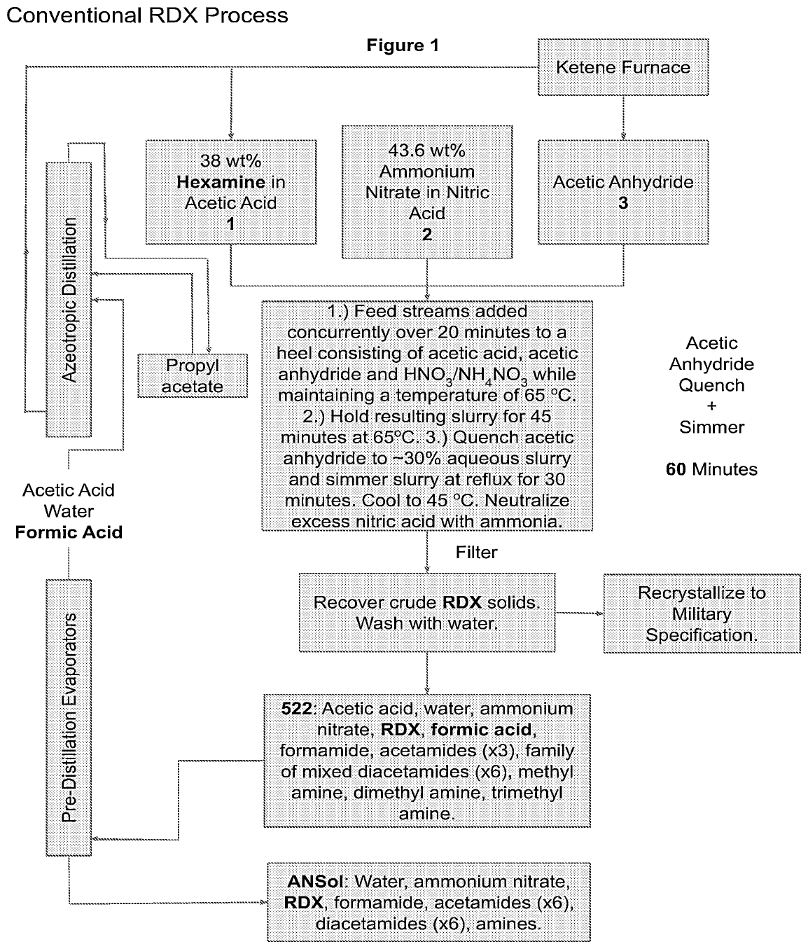

Referring to the flow diagram of the Hexahydro-1,3,5-trinitro-1,3,5-triazine nitrolysis shown in FIG. 1, the three feed streams which are entered into the reactor are as follows:
152.38 g of a solution of 38 wt % 1,3,5,7-tetraazatricyclo[3.3.1.1^{3,7}]decane in acetic acid,
217.35 g of a solution of 43.6 wt % ammonium nitrate in nitric acid,
330.63 g acetic anhydride.

The feed streams were added concurrently over 20 minutes to an agitated reactor containing a heel maintained at 65° C. and containing 451.4 g acetic acid, 21.9 g acetic anhydride, 11.8 g ammonium nitrate and 15.2 g nitric acid. Following the addition of the reagent feed streams the resulting slurry was aged at 65° C. for 45 minutes to ensure completion of the nitrolysis. Upon completion of the nitrolysis step 575 g water is added at a rate to ensure the reaction slurry temperature does not exceed 69° C. The resulting aqueous acetic acid slurry is heated to 98-100° C. for 30 minutes to destroy undesired linear nitramines. The slurry is cooled to 25° C. and filtered. The solids are washed with water and dried. The filtrate is processed through pre-distillation evaporators to separate the volatile components from the nonvolatile components. The process stream consisting mainly of acetic acid, water and methanoic acid are directed to the pre-distillation evaporators, followed by the distillation facility for final processing to glacial acetic acid (containing methanoic acid as a minor component) to complete the process cycle. A portion of the glacial acetic acid is directed toward the ketene furnaces to regenerate acetic anhydride and complete the process cycle.

Conventional Hexahydro-1,3,5-trinitro-1,3,5-triazine Process Modified According to the Present Invention

(Equivalent to the Hexahydro-1,3,5-trinitro-1,3,5-triazine Conventional Process through nitrolysis 45 minute age at 65° C.) Upon completion of the nitrolysis step at 65° C. for 45 minutes, the reaction slurry is cooled to 45° C. and treated with sufficient water to quench the acetic anhydride and adjust the water content to 0.0-35 wt % (minimum quantity of water required to consume the acetic anhydride and destroy the undesired linear nitramines and other hydrolysable, undesired side products during the simmer step). The resulting slurry is heated to 98-100° C. for 30 minutes followed by cooling to 45° C. To the slurry is added sufficient ammonia (ammonium acetate) to neutralize the excess nitric acid. The reaction slurry is maintained below 50° C. during the ammonia addition. To the neutralized slurry is added sufficient acetic anhydride to consume the water and adjust the acetic anhydride content to 0.0-0.50 wt %. The temperature is maintained below 65° C. during the acetic anhydride addition. The final slurry is cooled to 25° C. and filtered to the extent the majority of the acetic acid is recovered. The recovered solids are washed with water then recrystallized via the conventional process. The anhydrous spent acid is directed to the pre-distillation evaporators to separate the volatile components from the nonvolatile components. An appropriate portion of the glacial acetic acid recovered from the evaporators (containing 0.0-0.50% acetic anhydride) is directed to the ketene furnaces to regenerate acetic anhydride to complete the recycle process. An appropriate portion of the glacial acetic acid recovered from the evaporators (containing 0.0-0.50% acetic anhydride) is directed toward the dissolution of 1,3,5,7-tetraazatricyclo[3.3.1.1^{3,7}]decane. The remaining glacial acetic acid recovered from the evaporators (containing 0.0-0.50% acetic anhydride) is directed to the heel of the subsequent nitrolysis batch. The ANSol recovered from the pre-distillation evaporators can be recycled to the initial crude Hexahydro-1,3,5-trinitro-1,3,5-triazine filtration.

Conventional Hexahydro-1,3,5-trinitro-1,3,5-triazine Process Modified According to the Present Invention—Direct Recycle of Spent Acid

(Equivalent to the Hexahydro-1,3,5-trinitro-1,3,5-triazine Conventional Process through nitrolysis 45 minute age at 65° C.) Upon completion of the nitrolysis step at 65° C. for 45 minutes, the reaction slurry is cooled to 45° C. and treated with sufficient water to quench the acetic anhydride and adjust the water content to 0.0-35 wt % (minimum quantity of water required to consume the acetic anhydride and destroy the undesired linear nitramines and other hydrolysable, undesired side products during the simmer step). The resulting slurry is heated to 98-100° C. for 30 minutes followed by cooling to 45° C. To the slurry is added sufficient ammonia (ammonium acetate) to neutralize the excess nitric acid. The reaction slurry is maintained below 50° C. during the ammonia addition. To the neutralized slurry is added sufficient acetic anhydride to consume the water and adjust the acetic anhydride content to 0.0-0.50 wt %. The temperature is maintained below 65° C. during the acetic anhydride addition. The final slurry is cooled to 25° C. and filtered to the extent the majority of the acetic acid is recovered. The recovered solids are washed with water then recrystallized via the conventional process. The anhydrous spent acid is recycled directly to the subsequent Hexahydro-1,3,5-trinitro-1,3,5-triazine nitration heel and 1,3,5,7-tetraazatricyclo[3.3.1.1^{3,7}]decane dissolution. The ammonium nitrate content in the recycled spent acid is determined and accounted for when creating the subsequent nitration heel. The amount of ammonium nitrate added when creating the heel is decreased to account for the ammonium nitrate added via the spent acid. The remaining spent acid is sent to the pre-distillation evaporators to separate the volatile components from the nonvolatile components. An appropriate portion of the glacial acetic acid recovered from the evaporators (containing 0.0-0.50% acetic anhydride) is directed to the ketene furnaces to regenerate acetic anhydride to complete the recycle process. The ANSol recovered from the pre-distillation evaporators can be recycled to the initial crude hexahydro-1,3,5-trinitro-1,3,5-triazine filtration.
Example (assuming 1.0 wt % ammonium nitrate recycled acetic acid): 451.4 g acetic acid added to the heel. 451.4*0.01=4.51 g ammonium nitrate (note that the asterisk indicates a multiplication and is used at other places similarly in this document). 152.4 g of a 38 wt % 1,3,5,7-tetraazatricyclo[3.3.1.13,7]decane solution in acetic acid added via the feed stream. 0.62*152.4*0.01=0.94 g ammonium nitrate. 5.45 g (0.067 mol) total ammonium nitrate added to the heel from recycled acetic acid and 1,3,5,7-tetraazatricyclo[3.3.1.13,7]decane feed stream. Standard heel contains 11.8 g ammonium nitrate. 11.80−5.45=6.35 g ammonium nitrate to be added to the recycled heel to maintain concentrations of a standard heel.
   Conventional Hexahydro-1,3,5-trinitro-1,3,5-triazine Process Modified According to the Present Invention—Restore Anhydrous State—Spent Acid to the Pre-Distillation Evaporators

(Equivalent to the hexahydro-1,3,5-trinitro-1,3,5-triazine Conventional Process through nitrolysis 45 minute age at 65° C.). Upon completion of the nitrolysis step at 65° C. for 45 minutes, the reaction slurry is cooled to 45° C. (25-55° C.) and treated with sufficient ammonia (ammonium acetate) to neutralize the excess nitric acid. Neutralization step can happen after filtration, and before evaporators. The reaction slurry is maintained below 60° C. (40-60° C.) during the ammonia addition. To the neutralized slurry is added sufficient water to consume the acetic anhydride and adjust the water content to 0.0-35 wt %. The temperature is maintained below 60° C. (40-60° C.) during the water addition. The aqueous slurry is treated with sufficient acetic anhydride to consume the water and adjust the final acetic anhydride content to 0.0-0.50 wt % acetic anhydride. The final slurry is cooled to 25° C. (20-50° C.) and filtered to the extent the majority of the acetic acid is recovered. No simmer step is required. The filtered solids are stirred in hot (>90° C.) 0.0-90% nitric acid and heated to reflux, and the new slurry stirred for 15-60 minutes at reflux, followed by cooling to 25° C. (20-60° C.) and filtering. The recovered solids are washed with water then recrystallized via the conventional process. The resulting solution recovered from the hot 0.0-90% nitric acid slurry of the filtered solids is recycled to subsequent crude filtered solids batch treatments. The anhydrous spent acid is directed to the pre-distillation evaporators to separate the volatile components from the nonvolatile components. An appropriate portion of the glacial acetic acid recovered from the pre-distillation evaporators (containing 0.0-0.50% acetic anhydride) is directed to the ketene furnaces to regenerate acetic anhydride to complete the recycle process. An appropriate portion of the glacial acetic acid recovered from the pre-distillation evaporators (containing 0.0-0.50% acetic anhydride) is directed toward the dissolution of 1,3,5,7-tetraazatricyclo[3.3.1.1^{3,7}]decane. The remaining glacial acetic acid recovered from the pre-distillation evaporators (containing 0.0-0.50% acetic anhydride) is directed to the heel of the subsequent nitrolysis batch. The ANSol recovered from the pre-distillation evaporators can be recycled to the initial crude hexahydro-1,3,5-trinitro-1,3,5-triazine filtration.

Alternatively, the process may be returned to anhydrous conditions after filtration of solids. (Equivalent to the hexahydro-1,3,5-trinitro-1,3,5-triazine Conventional Process through nitrolysis 45 minute age at 65° C.). Upon completion of the nitrolysis step at 65° C. for 45 minutes, the reaction slurry is cooled to 45° C. (25-55° C.) and treated with sufficient ammonia (ammonium acetate) to neutralize the excess nitric acid. Neutralization step can happen after filtration, and before evaporators. The reaction slurry is maintained below 60° C. (40-60° C.) during the ammonia addition. To the neutralized slurry is added sufficient water to consume the acetic anhydride and adjust the water content to 0.0-35 wt %. The temperature is maintained below 60° C. (40-60° C.) during the water addition. The final slurry is cooled to 25° C. (20-50° C.) and filtered to the extent the majority of the acetic acid is recovered. No simmer step is required. The filtered solids are stirred in hot (>90° C.) 0.0-90% nitric acid and heated to reflux, and the new slurry stirred for 15-60 minutes at reflux, followed by cooling to 25° C. (20-60° C.) and filtering. The aqueous filtrate is treated with sufficient acetic anhydride to consume the water and adjust the final acetic anhydride content to 0.0-0.50 wt % acetic anhydride. The recovered solids are washed with water then recrystallized via the conventional process. The resulting solution recovered from the hot 0.0-90% nitric acid slurry of the filtered solids is recycled to subsequent crude filtered solids batch treatments. The anhydrous spent acid is directed to the pre-distillation evaporators to separate the volatile components from the nonvolatile components. An appropriate portion of the glacial acetic acid recovered from the pre-distillation evaporators (containing 0.0-0.50% acetic anhydride) is directed to the ketene furnaces to regenerate acetic anhydride to complete the recycle process. An appropriate portion of the glacial acetic acid recovered from the pre-distillation evaporators (containing 0.0-0.50% acetic anhydride) is directed toward the dissolution of 1,3,5,7-tetraazatricyclo[3.3.1.1^{3,7}]decane. The remaining glacial acetic acid recovered from the pre-distillation evaporators (containing 0.0-0.50% acetic anhydride) is directed to the heel of the subsequent nitrolysis batch. The ANSol recovered from the pre-distillation evaporators can be recycled to the initial crude hexahydro-1,3,5-trinitro-1,3,5-triazine filtration.

Direct Recycle of Spent Acid Hexahydro-1,3,5-trinitro-1,3,5-triazine Modification with Quench—Restore Anhydrous State—Recycle Spent Acid to Heel and 1,3,5,7-tetraazatricyclo[3.3.1.1^{3,7}]decane

(Equivalent to the hexahydro-1,3,5-trinitro-1,3,5-triazine the Conventional Process through nitrolysis 45 minute age at 65° C.) Upon completion of the nitrolysis step at 65° C. for 45 minutes, the reaction slurry is cooled to 45° C. (25-55° C.) and treated with sufficient ammonia (ammonium acetate) to neutralize the excess nitric acid. (Neutralization step can happen after filtration, and before evaporators.) The reaction slurry is maintained below 60° C. (40-60° C.) during the ammonia addition. To the neutralized slurry is added sufficient water to consume the acetic anhydride and adjust the water content to 0.0-35 wt %. The temperature is maintained below 60° C. (40-60° C.) during the water addition. The aqueous slurry is treated with sufficient acetic anhydride to consume the water and adjust the final acetic anhydride content to 0.0-0.50 wt % acetic anhydride. The final slurry is cooled to 25° C. (20-50° C.) and filtered to the extent the majority of the acetic acid is recovered. No simmer step is required. The filtered solids are stirred in hot (>90° C.) 0.0-90% nitric acid and heated to reflux, and the new slurry stirred for 15-60 minutes at reflux, followed by cooling to 25° C. (20-60° C.) and filtering. The recovered solids are washed with water then recrystallized via the conventional process. The resulting solution recovered from the hot 0.0-90% nitric acid slurry of the filtered solids is recycled to subsequent crude filtered solids batch treatments. An appropriate portion of the anhydrous spent acid (with 0.0-0.50% acetic anhydride and ˜1.0-1.5 wt % ammonium nitrate present) is directed to the dissolution of 1,3,5,7-tetraazatricyclo[3.3.1.1^{3,7}]decane and the heel of the subsequent nitration batch. The amount of ammonium nitrate present (from the heel and 1,3,5,7-tetraazatricyclo[3.3.1.1^{3,7} feed stream) is to be calculated and accounted for when setting the starting heel conditions. The remaining spent acid is sent to the pre-distillation evaporators to separate the volatile components from the nonvolatile components. An appropriate portion of the glacial acetic acid recovered from the evaporators (containing 0.0-0.50% acetic anhydride) is directed to the ketene furnaces to regenerate acetic anhydride to complete the recycle process. The ANSol recovered from the pre-distillation evaporators can be recycled to the initial crude hexahydro-1,3,5-trinitro-1,3,5-triazine filtration.

Alternatively, the process may be returned to anhydrous conditions after filtration of solids. (Equivalent to the hexahydro-1,3,5-trinitro-1,3,5-triazine the Conventional Process through nitrolysis 45 minute age at 65° C.) Upon completion of the nitrolysis step at 65° C. for 45 minutes, the reaction slurry is cooled to 45° C. (25-55° C.) and treated with sufficient ammonia (ammonium acetate) to neutralize the excess nitric acid. (Neutralization step can happen after filtration, and before evaporators.) The reaction slurry is maintained below 60° C. (40-60° C.) during the ammonia addition. To the neutralized slurry is added sufficient water to consume the acetic anhydride and adjust the water content to 0.0-35 wt %. The temperature is maintained below 60° C. (40-60° C.) during the water addition. The final slurry is cooled to 25° C. (20-50° C.) and filtered to the extent the majority of the acetic acid is recovered. No simmer step is required. The aqueous filtrate is treated with sufficient acetic anhydride to consume the water and adjust the final acetic anhydride content to 0.0-0.50 wt % acetic anhydride. The filtered solids are stirred in hot (>90° C.) 0.0-90% nitric acid and heated to reflux, and the new slurry stirred for 15-60 minutes at reflux, followed by cooling to 25° C. (20-60° C.) and filtering. The recovered solids are washed with water then recrystallized via the conventional process. The resulting solution recovered from the hot 0.0-90% nitric acid slurry of the filtered solids is recycled to subsequent crude filtered solids batch treatments. An appropriate portion of the anhydrous spent acid (with 0.0-0.50% acetic anhydride and ˜1.0-1.5 wt % ammonium nitrate present) is directed to the dissolution of 1,3,5,7-tetraazatricyclo[3.3.1.1^{3,7}]decane and the heel of the subsequent nitration batch. The amount of ammonium nitrate present (from the heel and 1,3,5,7-tetraazatricyclo[3.3.1.1^{3,7} feed stream) is to be calculated and accounted for when setting the starting heel conditions. The remaining spent acid is sent to the pre-distillation evaporators to separate the volatile components from the nonvolatile components. An appropriate portion of the glacial acetic acid recovered from the evaporators (containing 0.0-0.50% acetic anhydride) is directed to the ketene furnaces to regenerate acetic anhydride to complete the recycle process. The ANSol recovered from the pre-distillation evaporators can be recycled to the initial crude hexahydro-1,3,5-trinitro-1,3,5-triazine filtration.
Example (assuming 1.0 wt % ammonium nitrate recycled acetic acid): 451.4 g acetic acid added to the heel. 451.4*0.01=4.51 g ammonium nitrate. 152.4 g of a 38 wt % 1,3,5,7-tetraazatricyclo[3.3.1.1^{3,7}]decane solution in acetic acid added via the feed stream. 0.62*152.4*0.01=0.94 g ammonium nitrate. 5.45 g (0.067 mol) total ammonium nitrate added to the heel from recycled acetic acid and 1,3,5,7-tetraazatricyclo[3.3.1.1^{3,7}]decane feed stream. Standard heel contains 11.8 g ammonium nitrate. 11.80−5.45=6.35 g ammonium nitrate to be added to the recycled heel.
   Conventional Process Octahydro-1,3,5,7-tetranitro-1,3,5,7-tetrazocine

Referring to the flow diagram of the Octahydro-1,3,5,7-tetranitro-1,3,5,7-tetrazocine nitrolysis shown in the drawing, the multiphase additions are entered into the reactor as follows:

To a heel containing 437.6 g acetic acid and 10.9 g acetic anhydride are added three feedstreams concurrently:
152.4 g of a 38 wt % 1,3,5,7-tetraazatricyclo[3.3.1.1^{3,7}]decane solution in acetic acid,
94.9 g of a 43.6 wt % ammonium nitrate solution in nitric acid,
325.5 g of acetic anhydride.

The feedstreams were added concurrently over 20 minutes to an agitated reactor containing a heel maintained at 44° C. Upon complete addition, the reaction slurry is aged at 44° C. for 6 minutes.

The reactor containing the first stage slurry is treated with two feedstreams added concurrently over 7 minutes while maintaining a reactor temperature of 44° C.:
26.9 g of 43.6 wt % ammonium nitrate solution in nitric acid,
96.9 g acetic anhydride.

Immediately following the stage two addition, the reactor containing the second stage slurry is treated with two feedstreams added concurrently over 8 minutes while maintaining a reactor temperature of 44° C.:
124.0 g 43.6 wt % ammonium nitrate in nitric acid,
352.3 g acetic anhydride.

Following the addition of the reagent feedstreams the resulting slurry was aged at 44° C. for 45 minutes to ensure completion of the nitrolysis. Upon completion of the nitrolysis step 718.8 g water is added at a rate to ensure the reaction slurry temperature does not exceed 50° C. The resulting aqueous acetic acid slurry is heated to 98-100° C. for 120 minutes to destroy undesired linear nitramines. The slurry is cooled to 40° C. and filtered. The solids are washed with water and dried. The filtrate is processed through pre-distillation evaporators to separate the volatile components from the nonvolatile components. The process stream consisting mainly of acetic acid, water and methanoic acid are directed to the distillation facility for final processing to glacial acetic acid (containing 0.20 to 0.40 wt % methanoic acid) to complete the process cycle. A portion of the glacial acetic acid is directed toward the ketene furnaces to regenerate acetic anhydride and complete the process cycle.

Conventional Octahydro-1,3,5,7-tetranitro-1,3,5,7-tetrazocine Process Modified According to the Present Invention

(Equivalent to the Conventional Octahydro-1,3,5,7-tetranitro-1,3,5,7-tetrazocine Process to this point). Upon completion of the nitrolysis age step at 44° C. for 45 minutes, the reaction slurry is treated with sufficient water to quench the acetic anhydride and adjust the water content to 0.0-20 wt % aqueous acetic acid (minimum quantity of water required to consume the acetic anhydride and destroy the undesired linear nitramines and other hydrolysable, undesired side products during the simmer step). The resulting slurry is heated to 98-100° C. for 120 minutes followed by cooling to 45° C. To the slurry is added sufficient ammonia (ammonium acetate) to neutralize the excess nitric acid. The reaction slurry is maintained below 50° C. during the ammonia addition. To the neutralized slurry is added sufficient acetic anhydride to consume the water and adjust the acetic anhydride content to 0.0-0.50 wt %. The temperature is maintained below 65° C. during the acetic anhydride addition. The final slurry is heated to 50-90° C. (a temperature sufficient to extract the RDX content to <2.0% of the HMX content) and filtered to the extent the majority of the acetic acid is recovered. The recovered solids are washed with water then recrystallized via the conventional process. The anhydrous spent acid is directed to the pre-distillation evaporators to separate the volatile components from the nonvolatile components. An appropriate portion of the glacial acetic acid recovered from the evaporators (containing 0.0-0.50% acetic anhydride) is directed to the ketene furnaces to regenerate acetic anhydride to complete the recycle process. An appropriate portion of the glacial acetic acid recovered from the evaporators (containing 0.0-0.50% acetic anhydride) is directed toward the dissolution of 1,3,5,7-tetraazatricyclo[3.3.1.1^{3,7}]decane. The remaining glacial acetic acid recovered from the evaporators (containing 0.0-0.50% acetic anhydride) is directed to the heel of the subsequent nitrolysis batch. The ANSol recovered from the pre-distillation evaporators can be recycled to the crude Hexahydro-1,3,5-trinitro-1,3,5-triazine filtration.

Conventional Octahydro-1,3,5,7-tetranitro-1,3,5,7-tetrazocine Process Modified According to the Present Invention—Direct Recycle of Spent Acid

(Equivalent to the Conventional Octahydro-1,3,5,7-tetranitro-1,3,5,7-tetrazocine Process to this point utilizing methanoic acid free solvents). Upon completion of the nitrolysis age step at 44° C. for 45 minutes, the reaction slurry is treated with sufficient water to quench the acetic anhydride and adjust the water content to 0.0-20 wt % aqueous acetic acid (minimum quantity of water required to consume the acetic anhydride and destroy the undesired linear nitramines and other hydrolysable, undesired side products during the simmer step). The resulting slurry is heated to 98-100° C. for 120 minutes followed by cooling to 45° C. To the slurry is added sufficient ammonia (ammonium acetate) to neutralize the excess nitric acid. The reaction slurry is maintained below 50° C. during the ammonia addition. To the neutralized slurry is added sufficient acetic anhydride to consume the water and adjust the acetic anhydride content to 0.0-0.50 wt %. The temperature is maintained below 65° C. during the acetic anhydride addition. The final slurry is heated to 50-90° C. (a temperature sufficient to extract the RDX content to <2.0% of the HMX content) and filtered to the extent the majority of the acetic acid is recovered. The recovered solids are washed with water then recrystallized via the conventional process. The anhydrous spent acid is cooled to 25° C. and filtered again. The filtered anhydrous spent acid is recycled directly to the subsequent Octahydro-1,3,5,7-tetranitro-1,3,5,7-tetrazocine nitration heel and 1,3,5,7-tetraazatricyclo[3.3.1.1^{3,7}]decane dissolution. The ammonium nitrate content in the recycled spent acid is determined (x moles) and accounted for when creating the subsequent Octahydro-1,3,5,7-tetranitro-1,3,5,7-tetrazocine nitration heel. To the subsequent spent acid nitration heel at 20-25° C., with x moles of ammonium nitrate, is added 0.50x-1.0x moles of 1,3,5,7-tetraazatricyclo[3.3.1.1^{3,7}]decane and x moles of nitric acid while maintaining the temperature below 30° C. Alternatively, if the Hexahydro-1,3,5-trinitro-1,3,5-triazine content of the recycled spent acid is too high and the Military Specification for the subsequent Octahydro-1,3,5,7-tetranitro-1,3,5,7-tetrazocine batches is difficult to achieve (<2.0 wt % Hexahydro-1,3,5-trinitro-1,3,5-triazine filtration), the anhydrous spent acid is recycled to a subsequent Hexahydro-1,3,5-trinitro-1,3,5-triazine nitration step. The remaining spent acid is sent to the pre-distillation evaporators to separate the volatile components from the nonvolatile components. An appropriate portion of the glacial acetic acid recovered from the evaporators (containing 0.0-0.50% acetic anhydride) is directed to the ketene furnaces to regenerate acetic anhydride to complete the recycle process. The ANSol recovered from the pre-distillation evaporators can be recycled to the initial crude hexahydro-1,3,5-trinitro-1,3,5-triazine filtration.
Example (assuming 1.0 wt % ammonium nitrate recycled acetic acid): 437.6 g acetic acid added to the heel. 437.6*0.01=4.38 g ammonium nitrate. 152.4 g of a 38 wt % 1,3,5,7-tetraazatricyclo[3.3.1.1^{3,7}]decane solution in acetic acid added via the feed stream. 0.62*152.4*0.01=0.94 g ammonium nitrate. 5.32 g (0.067 mol) total ammonium nitrate added to the heel/reaction. 4.22 g (0.067 mol) nitric acid and 4.69-9.38 g (0.034-0.067 mol) 1,3,5,7-tetraazatricyclo[3.3.1.1^{3,7}]decane added to the recycled heel.
   Conventional Octahydro-1,3,5,7-tetranitro-1,3,5,7-tetrazocine Process Modified According to the Present Invention—Restore Anhydrous State—Spent Acid to the Pre-Distillation Evaporators

(Equivalent to the Conventional Octahydro-1,3,5,7-tetranitro-1,3,5,7-tetrazocine Process through nitrolysis 45 minute age at 44° C.) Upon completion of the nitrolysis age step at 44° C. for 45 minutes, the reaction slurry is treated with sufficient ammonia (or ammonium acetate) to neutralize the excess nitric acid. The reaction slurry is maintained below 60° C. (40-60° C.) during the ammonia addition. To the neutralized slurry is added sufficient water to consume the acetic anhydride and adjust the water content to 0.0-35 wt %. The temperature is maintained below 60° C. (40-60° C.) during the water addition. The aqueous slurry is treated with sufficient acetic anhydride to consume the water and adjust the final acetic anhydride content to 0.0-0.50 wt % acetic anhydride. The final slurry is filtered at 50-90° C. (a temperature sufficient to extract the RDX content to <2.0% of the HMX content) to the extent the majority of the acetic acid is recovered. No simmer step is required. The filtered solids are stirred in hot (>90° C.) 0.0-90% nitric acid heated to reflux, and the new refluxing slurry stirred for 15-60 minutes, followed by cooling to 60° C. (60-80° C.) and filtering. The recovered solids are washed with water then recrystallized via the conventional process. The resulting solution recovered from the hot 0.0-90% nitric acid slurry of the filtered solids is recycled to subsequent crude filtered solids batch treatments. The anhydrous spent acid is directed to the pre-distillation evaporators to separate the volatile components from the nonvolatile components. An appropriate portion of the glacial acetic acid recovered from the pre-distillation evaporators (containing 0.0-0.50% acetic anhydride) is directed to the ketene furnaces to regenerate acetic anhydride to complete the recycle process. An appropriate portion of the glacial acetic acid recovered from the pre-distillation evaporators (containing 0.0-0.50% acetic anhydride) is directed toward the dissolution of 1,3,5,7-tetraazatricyclo[3.3.1.1^{3,7}]decane. The remaining glacial acetic acid recovered from the pre-distillation evaporators (containing 0.0-0.50% acetic anhydride) is directed to the heel of the subsequent nitrolysis batch. The ANSol recovered from the pre-distillation evaporators can be recycled to the initial crude hexahydro-1,3,5-trinitro-1,3,5-triazine filtration.

Alternatively, the process may be returned to anhydrous conditions after filtration of solids. (Equivalent to the Conventional Octahydro-1,3,5,7-tetranitro-1,3,5,7-tetrazocine Process through nitrolysis 45 minute age at 44° C.) Upon completion of the nitrolysis age step at 44° C. for 45 minutes, the reaction slurry is treated with sufficient ammonia (or ammonium acetate) to neutralize the excess nitric acid. The reaction slurry is maintained below 60° C. (40-60° C.) during the ammonia addition. To the neutralized slurry is added sufficient water to consume the acetic anhydride and adjust the water content to 0.0-35 wt %. The temperature is maintained below 60° C. (40-60° C.) during the water addition. The final slurry is filtered at 50-90° C. (a temperature sufficient to extract the RDX content to <2.0% of the HMX content) to the extent the majority of the acetic acid is recovered. No simmer step is required. The aqueous filtrate is treated with sufficient acetic anhydride to consume the water and adjust the final acetic anhydride content to 0.0-0.50 wt % acetic anhydride. The filtered solids are stirred in hot (>90° C.) 0.0-90% nitric acid heated to reflux, and the new refluxing slurry stirred for 15-60 minutes, followed by cooling to 60° C. (60-80° C.) and filtering. The recovered solids are washed with water then recrystallized via the conventional process. The resulting solution recovered from the hot 0.0-90% nitric acid slurry of the filtered solids is recycled to subsequent crude filtered solids batch treatments. The anhydrous spent acid is directed to the pre-distillation evaporators to separate the volatile components from the nonvolatile components. An appropriate portion of the glacial acetic acid recovered from the pre-distillation evaporators (containing 0.0-0.50% acetic anhydride) is directed to the ketene furnaces to regenerate acetic anhydride to complete the recycle process. An appropriate portion of the glacial acetic acid recovered from the pre-distillation evaporators (containing 0.0-0.50% acetic anhydride) is directed toward the dissolution of 1,3,5,7-tetraazatricyclo[3.3.1.1^{3,7}]decane. The remaining glacial acetic acid recovered from the pre-distillation evaporators (containing 0.0-0.50% acetic anhydride) is directed to the heel of the subsequent nitrolysis batch. The ANSol recovered from the pre-distillation evaporators can be recycled to the initial crude hexahydro-1,3,5-trinitro-1,3,5-triazine filtration.

Direct Recycle of Spent Acid Octahydro-1,3,5,7-tetranitro-1,3,5,7-tetrazocine Modification—Restore Anhydrous State—Recycle Spent Acid to Heel and 1,3,5,7-tetraazatricyclo[3.3.1.1^{3,7}]decane

(Equivalent to the Conventional Octahydro-1,3,5,7-tetranitro-1,3,5,7-tetrazocine Process through nitrolysis 45 minute age at 44° C.) Upon completion of the nitrolysis age step at 44° C. for 45 minutes, the reaction slurry is treated with sufficient ammonia (or ammonium acetate) to neutralize the excess nitric acid. The reaction slurry is maintained below 60° C. (40-60° C.) during the ammonia addition. To the neutralized slurry is added sufficient water to consume the acetic anhydride and adjust the water content to 0.0-35 wt %. The temperature is maintained below 60° C. (40-60° C.) during the water addition. The aqueous slurry is treated with sufficient acetic anhydride to consume the water and adjust the final acetic anhydride content to 0.0-0.50 wt % acetic anhydride. The final slurry is warmed to 50-90° C. (a temperature sufficient to extract the RDX content to <2.0% of the HMX content) and filtered to the extent the majority of the acetic acid is recovered. No simmer step is required. The filtered solids are stirred in hot (>90° C.) 0.0-90% nitric acid and heated to reflux, and the new refluxing slurry stirred for 15-60 minutes, followed by cooling to 60° C. (60-80° C.) and filtering. The recovered solids are washed with water then recrystallized via the conventional process. The resulting solution recovered from the hot 0.0-90% nitric acid slurry of the filtered solids is recycled to subsequent crude filtered solids batch treatments. The spent acid is cooled to 20° C. and filtered. The spent acid contains ammonium nitrate (˜1.0-1.50 wt %). An appropriate portion of the anhydrous spent acid is directed toward dissolution of 1,3,5,7-tetraazatricyclo[3.3.1.1^{3,7}]decane. An appropriate portion of the anhydrous spent acid is directed toward the heel of the subsequent nitrolysis batch. The total ammonium nitrate from the 1,3,5,7-tetraazatricyclo[3.3.1.1^{3,7}]decane feed and heel formation carried into the subsequent nitration step is calculated. An equal molar amount (relative to the amount of ammonium nitrate present in the heel and 1,3,5,7-tetraazatricyclo[3.3.1.1^{3,7}]decane feed stream) of nitric acid is added to the heel. 0.50-1.0 molar equivalents (relative to the amount of ammonium nitrate present in the heel and 1,3,5,7-tetraazatricyclo[3.3.1.1^{3,7}]decane feed stream) of 1,3,5,7-tetraazatricyclo[3.3.1.1^{3,7}]decane are added to the heel. The remaining spent acid is directed to the pre-distillation evaporators to separate volatile components from nonvolatile components. The pre-distilled spent acid is directed to the ketene furnaces to regenerate acetic anhydride to complete the recycle process.

Alternatively, the process may be returned to anhydrous conditions after filtration of solids. (Equivalent to the Conventional Octahydro-1,3,5,7-tetranitro-1,3,5,7-tetrazocine Process through nitrolysis 45 minute age at 44° C.) Upon completion of the nitrolysis age step at 44° C. for 45 minutes, the reaction slurry is treated with sufficient ammonia (or ammonium acetate) to neutralize the excess nitric acid. The reaction slurry is maintained below 60° C. (40-60° C.) during the ammonia addition. To the neutralized slurry is added sufficient water to consume the acetic anhydride and adjust the water content to 0.0-35 wt %. The temperature is maintained below 60° C. (40-60° C.) during the water addition. The final slurry is warmed to 50-90° C. (a temperature sufficient to extract the RDX content to <2.0% of the HMX content) and filtered to the extent the majority of the acetic acid is recovered. No simmer step is required. The aqueous filtrate is treated with sufficient acetic anhydride to consume the water and adjust the final acetic anhydride content to 0.0-0.50 wt % acetic anhydride. The filtered solids are stirred in hot (>90° C.) 0.0-90% nitric acid and heated to reflux, and the new refluxing slurry stirred for 15-60 minutes, followed by cooling to 60° C. (60-80° C.) and filtering. The recovered solids are washed with water then recrystallized via the conventional process. The resulting solution recovered from the hot 0.0-90% nitric acid slurry of the filtered solids is recycled to subsequent crude filtered solids batch treatments. The spent acid is cooled to 20° C. and filtered. The spent acid contains ammonium nitrate (˜1.0-1.50 wt %). An appropriate portion of the anhydrous spent acid is directed toward dissolution of 1,3,5,7-tetraazatricyclo[3.3.1.1^{3,7}]decane. An appropriate portion of the anhydrous spent acid is directed toward the heel of the subsequent nitrolysis batch. The total ammonium nitrate from the 1,3,5,7-tetraazatricyclo[3.3.1.1^{3,7}]decane feed and heel formation carried into the subsequent nitration step is calculated. An equal molar amount (relative to the amount of ammonium nitrate present in the heel and 1,3,5,7-tetraazatricyclo[3.3.1.1^{3,7}]decane feed stream) of nitric acid is added to the heel. 0.50-1.0 molar equivalents (relative to the amount of ammonium nitrate present in the heel and 1,3,5,7-tetraazatricyclo[3.3.1.1^{3,7}]decane feed stream) of 1,3,5,7-tetraazatricyclo[3.3.1.1^{3,7}]decane are added to the heel. The remaining spent acid is directed to the pre-distillation evaporators to separate volatile components from nonvolatile components. The pre-distilled spent acid is directed to the ketene furnaces to regenerate acetic anhydride to complete the recycle process.

Alternatively, if the Hexahydro-1,3,5-trinitro-1,3,5-triazine content of the resultant Octahydro-1,3,5,7-tetranitro-1,3,5,7-tetrazocine batches is greater than required for the Military Specification (<2.0 wt % Hexahydro-1,3,5-trinitro-1,3,5-triazine in Octahydro-1,3,5,7-tetranitro-1,3,5,7-tetrazocine) the anhydrous spent acid recovered from the Octahydro-1,3,5,7-tetranitro-1,3,5,7-tetrazocine process may be recycled to a subsequent Hexahydro-1,3,5-trinitro-1,3,5-triazine nitration step and 1,3,5,7-tetraazatricyclo[3.3.1.1^{3,7}]decane dissolution. The ANSol recovered from the pre-distillation evaporators can be recycled to the initial crude hexahydro-1,3,5-trinitro-1,3,5-triazine filtration.
Example (assuming 1.0 wt % ammonium nitrate recycled acetic acid): 437.6 g acetic acid added to the heel. 437.6*0.01=4.38 g ammonium nitrate. 152.4 g of a 38 wt % 1,3,5,7-tetraazatricyclo[3.3.1.1^{3,7}]decane solution in acetic acid added via the feed stream. 0.62*152.4*0.01=0.94 g ammonium nitrate. 5.32 g (0.067 mol) total ammonium nitrate added to the heel/reaction. 4.22 g (0.067 mol) nitric acid and 4.69-9.38 g (0.034-0.067 mol) 1,3,5,7-tetraazatricyclo[3.3.1.1^{3,7}]decane added to the recycled heel. This is not applicable to processes run with virgin acetic acid.

What is claimed is:

1. A direct recycle of spent acid process for producing Hexahydro-1,3,5-trinitro-1,3,5-triazine, by nitrolysis of 1,3,5,7-tetraazatricyclo[3.3.1.13·7]decane, which comprises:
a.) while maintaining a temperature near 65° C., introducing a stream of 1,3,5,7-tetraazatricyclo[3.3.1.13·7]decane in acetic acid, a stream of ammonium nitrate in nitric acid, and a stream of acetic anhydride concurrently in proportions greater than is necessary to form Hexahydro-1,3,5-trinitro-1,3,5-triazine, into a heel containing a majority acetic acid with nitric acid, ammonium nitrate and acetic anhydride present in the heel;
b.) heating the resulting slurry to 65° C. for 45 minutes to affect nitrolysis of the 1,3,5,7-tetraazatricyclo[3.3.1.13·7]decane to produce Hexahydro-1,3,5-trinitro-1,3,5-triazine and a spent acid containing acetic acid, acetic anhydride, nitric acid, ammonium nitrate;
c.) adding water to said slurry to consume the acetic anhydride and adjust the water content to approximately 0.0-35.0 wt % aqueous acetic acid (minimum quantity of water required to consume the acetic anhydride and destroy the undesired linear nitramines and other hydrolysable, undesired side products during the subsequent simmer step d);
d.) heating (simmering) the said aqueous acetic acid slurry to 98-100° C. for approximately 30 minutes;
e.) cooling the simmered slurry to approximately 45° C.;
f.) adding acetic anhydride to the said simmered slurry to consume the water and adjust the acetic anhydride content to 0.0-0.50 wt % acetic anhydride;
g.) adding ammonia (ammonium acetate) to the anhydrous, crude reaction slurry sufficient to neutralize the nitric acid;
h.) separating the Hexahydro-1,3,5-trinitro-1,3,5-triazine and the ammonium nitrate from the anhydrous spent acid;
i.) recycling the anhydrous (0.0-0.50 wt % acetic anhydride) spent acid through the pre-distillation evaporators to complete the process cycle and supply 1,3,5,7-Tetraazatricyclo[3.3.1.13·7]decane dissolution, heel formation and ketene furnace conversion to acetic anhydride;
j.) washing the crude Hexahydro-1,3,5-trinitro-1,3,5-triazine solids with hot water;
k.) recrystallization of the Hexahydro-1,3,5-trinitro-1,3,5-triazine via conventional processes.

2. The process of claim 1, wherein a part of the 0.0-0.50 wt % acetic anhydride spent acid is recycled to the subsequent Hexahydro-1,3,5-trinitro-1,3,5-triazine nitration heel, part of the 0.0-0.50 wt % acetic anhydride spent acid is recycled to dissolve the 1,3,5,7-tetraazatricyclo[3.3.1.13·7]decane, part of the 0.0-0.50 wt % acetic anhydride spent acid is processed through the pre-distillation evaporators followed by conversion to acetic anhydride in the ketene furnace; and the ammonium nitrate content in the 1,3,5,7-tetraazatricyclo[3.3.1.13·7]decane feed stream and acetic acid heel accounted for, and adjustments made in the subsequent standard heel formation.

3. The process of claim 1, wherein the 0.0-35 wt % aqueous spent acid slurry at 45° C. is filtered and the 0.0-35 wt % aqueous spent acid filtrate is processed through the pre-distillation evaporators followed by azeotropic distillation to complete the acetic acid recovery cycle.

4. The process in claim 1, wherein the 0.0-0.50 wt % acetic anhydride spent acid (with formic acid present) is processed through the pre-distillation evaporators followed by azeotropic distillation (separate formic acid from acetic acid) to complete the acetic acid recovery cycle.

5. A direct recycle of spent acid process for producing Octahydro-1,3,5,7-tetranitro-1,3,5,7-tetrazocine, by nitrolysis of 1,3,5,7-tetraazatricyclo[3.3.1.13·7]decane, which comprises:
aa.) while maintaining a temperature near 44° C., introducing a stream of 1,3,5,7-tetraazatricyclo[3.3.1.13.7]decane in acetic acid, a stream of ammonium nitrate in nitric acid, and a stream of acetic anhydride concurrently in proportions greater than is necessary to form Octahydro-1,3,5,7-tetranitro-1,3,5,7-tetrazocine intermediate 1 (stage 1), into a heel containing a majority acetic acid with acetic anhydride present in the heel;
ab.) while maintaining a temperature near 44° C., introducing stream of ammonium nitrate in nitric acid and a stream of acetic anhydride concurrently in proportions greater than is necessary to a reaction mixture containing Octahydro-1,3,5,7-tetranitro-1,3,5,7-tetrazocine intermediate 1, to form Octahydro-1,3,5,7-tetranitro-1,3,5,7-tetrazocine intermediate 2 (stage 2);
ac.) while maintaining a temperature near 44° C., introducing stream of ammonium nitrate in nitric acid and a stream of acetic anhydride concurrently in proportions greater than is necessary to a reaction mixture containing Octahydro-1,3,5,7-tetranitro-1,3,5,7-tetrazocine intermediate 2, to form Octahydro-1,3,5,7-tetranitro-1,3,5,7-tetrazocine (stage 3)
ad.) heating the resulting slurry to 44° C. to affect nitrolysis of the Octahydro-1,3,5,7-tetranitro-1,3,5,7-tetrazocine intermediate to produce Octahydro-1,3,5,7-tetranitro-1,3,5,7-tetrazocine and a crude reaction slurry containing acetic acid, acetic anhydride, nitric acid, ammonium nitrate;
ae.) adding water to said slurry to consume the acetic anhydride and adjust the water content to approximately 0.0-20.0 wt % aqueous acetic acid (minimum quantity of water required to consume the acetic anhydride and destroy the undesired linear nitramines and other hydrolysable, undesired side products during the subsequent simmer step ag);
af.) heating (simmering) the said aqueous acetic acid slurry to 98-100° C. for approximately 120 minutes;
ag.) cooling the simmered slurry to approximately 45° C.;
ah.) adding acetic anhydride to the said simmered slurry to consume the water and adjust the acetic anhydride content to 0.0-0.50 wt % acetic anhydride;
ai.) adding ammonia (ammonium acetate) to the anhydrous, crude reaction slurry sufficient to neutralize the nitric acid;
aj.) heating the neutralized slurry to 50-90° C. (a temperature sufficient to extract the RDX content to <2.0% of the HMX content) and separating the Octahydro-1,3,5,7-tetranitro-1,3,5,7-tetrazocine and the ammonium nitrate from the spent acid;
ak.) cooling the anhydrous acetic acid filtrate from step (ak) to 25° C. and filtering precipitated solids;
al.) recycling the filtered, anhydrous (0.0-0.50 wt % acetic anhydride) spent acid through the pre-distillation evaporators to complete the process cycle and supply 1,3,5,7-tetraazatricyclo[3.3.1.1 3·7]decane dissolution, subsequent heel formation and ketene furnace conversion to acetic anhydride;
am.) washing the filter cake with hot water and separating the Octahydro-1,3,5,7-tetranitro-1,3,5,7-tetrazocine from the hot water ammonium nitrate solution.

6. The process of claim 5, wherein a part of the 0.0-0.50 wt % acetic anhydride spent acid is from step (al) recycled to the subsequent Octahydro-1,3,5,7-tetranitro-1,3,5,7-tetrazocine heel, part of the 0.0-0.50 wt % acetic anhydride spent acid is recycled to dissolve the 1,3,5,7-tetraazatricyclo[3.3.1.13·7]decane, part of the 0.0-0.50 wt % acetic anhydride spent acid is processed through the pre-distillation evaporators followed by conversion to acetic anhydride in the ketene furnace; the ammonium nitrate content is accounted for in the 1,3,5,7-tetraazatricyclo[3.3.1.13·7]decane acetic acid feed stream (x moles), and the subsequent nitrolysis heel; and 1.0x moles nitric acid and 0.50x-1.0x moles 1,3,5,7-tetraazatricyclo[3.3.1.13·7]decane added to subsequent Octahydro-1,3,5,7-tetranitro-1,3,5,7-tetrazocine nitration heel.

7. The process of claim 5, wherein a part of the 0.0-0.50 wt % acetic anhydride spent acid is recycled to the subsequent Hexahydro-1,3,5-trinitro-1,3,5-triazine nitration heel, part of the 0.0-0.50 wt % acetic anhydride spent acid is recycled to dissolve the 1,3,5,7-tetraazatricyclo[3.3.1.13·7]decane (to be employed in the Hexahydro-1,3,5-trinitro-1,3,5-triazine process), part of the 0.0-0.50 wt % acetic anhydride spent acid is processed through the pre-distillation evaporators followed by conversion to acetic anhydride in the ketene furnace; and the ammonium nitrate content in the 1,3,5,7-tetraazatricyclo[3.3.1.13·7]decane feed stream and acetic acid heel accounted for, and adjustments made in the standard heel formation.

8. The process of claim 5, wherein the 0.0-20 wt % aqueous spent acid slurry at 45° C. is filtered and the 0.0-20 wt % aqueous spent acid filtrate is processed through the pre-distillation evaporators followed by azeotropic distillation to complete the acetic acid recovery cycle.

9. The process according to claim 1 in which the amount of water added in steps c or ae is titrated to consume the linear nitramines and excess acetic anhydride and produce an anhydrous spent acid stream following the 98-100° C. simmer step (steps d and af).

10. The process in claim 5, wherein the 0.0-0.50 wt % acetic anhydride spent acid (separate formic acid from acetic acid) is processed through the pre-distillation evaporators followed by azeotropic distillation to complete the acetic acid recovery cycle.

11. The process of claim 6, wherein the anhydrous spent acid is recycled to a subsequent Hexahydro-1,3,5-trinitro-1,3,5-triazine nitration step.

12. A direct recycle of spent acid process for producing Hexahydro-1,3,5-trinitro1,3,5-triazine, by nitrolysis of 1,3,5,7-tetraazatricyclo[3.3.1.13·7]decane, which comprises:
ba.) while maintaining a temperature near 65° C., introducing a stream of 1,3,5,7-tetraazatricyclo[3.3.1.13·7]decane in acetic acid, a stream of ammonium nitrate in nitric acid, and a stream of acetic anhydride concurrently in proportions greater than is necessary to form Hexahydro-1,3,5-trinitro-1,3,5-triazine, into a heel containing a majority acetic acid with nitric acid, ammonium nitrate and acetic anhydride present in the heel;
bb.) heating the resulting slurry to 65° C. for 45 minutes to affect nitrolysis of the 1,3,5,7-tetraazatricyclo[3.3.1.13·7]decane to produce Hexahydro-1,3,5-trinitro1,3,5-triazine and a spent acid containing acetic acid, acetic anhydride, nitric acid, ammonium nitrate;
bc.) adding water to said slurry to consume the acetic anhydride and adjust the water content to approximately 0.0-35.0 wt % aqueous acetic acid;
bd.) cooling the slurry to approximately 45° C.;
be.) adding acetic anhydride to the said slurry to consume the water and adjust the acetic anhydride content to 0.0-0.50 wt % acetic anhydride;
bf.) adding ammonia (ammonium acetate) to the anhydrous, crude reaction slurry sufficient to neutralize the nitric acid;
bg.) separating the Hexahydro-1,3,5-trinitro-1,3,5-triazine and the ammonium nitrate from the anhydrous spent acid;
bh.) stirring the hexahydro-1,3,5-trinitro-1,3,5-triazine from step (bg) in a 0.0-90 wt % nitric acid solution at reflux to destroy undesired linear nitramines to provide crude hexahydro-1,3,5-trinitro-1,3,5-triazine;
bi.) collecting the crude hexahydro-1,3,5-trinitro-1,3,5-triazine from step (bh) by filtration;
bj.) recycling the anhydrous (0.0-0.50 wt % acetic anhydride) spent acid through the pre-distillation evaporators to complete the process cycle and supply 1,3,5,7-Tetraazatricy clo[3.3.1.13·7]decane dissolution, heel formation and ketene furnace conversion to acetic anhydride;
bk.) washing the crude Hexahydro-1,3,5-trinitro-1,3,5-triazine solids with hot water; and
bl.) recrystallization of the Hexahydro-1,3,5-trinitro-1,3,5-triazine via conventional processes.

13. A direct recycle of spent acid process for producing Hexahydro-1,3,5-trinitro1,3,5-triazine, by nitrolysis of 1,3,5,7-tetraazatricyclo[3.3.1.13·7]decane, which comprises:
ca.) while maintaining a temperature near 65° C., introducing a stream of 1,3,5,7-tetraazatricyclo[3.3.1.13·7]decane in acetic acid, a stream of ammonium nitrate in nitric acid, and a stream of acetic anhydride concurrently in proportions greater than is necessary to form Hexahydro-1,3,5-trinitro-1,3,5-triazine, into a heel containing a majority acetic acid with nitric acid, ammonium nitrate and acetic anhydride present in the heel;
cb.) heating the resulting slurry to 65° C. for 45 minutes to affect nitrolysis of the 1,3,5,7-tetraazatricyclo[3.3.1.13·7]decane to produce Hexahydro-1,3,5-trinitro1,3,5-triazine and a spent acid containing acetic acid, acetic anhydride, nitric acid, ammonium nitrate;
cc.) adding water to said slurry to consume the acetic anhydride and adjust the water content to approximately 0.0-35.0 wt % aqueous acetic acid;
cd.) cooling the slurry to approximately 45° C.;
ce.) adding ammonia (ammonium acetate) to the anhydrous, crude reaction slurry sufficient to neutralize the nitric acid;
cf.) separating the Hexahydro-1,3,5-trinitro-1,3,5-triazine and the ammonium nitrate from the anhydrous spent acid;
cg.) adding acetic anhydride to the said filtrate to consume the water and adjust the acetic anhydride content to 0.0-0.50 wt % acetic anhydride;
ch.) stirring the hexahydro-1,3,5-trinitro-1,3,5-triazine from step (cg) in a 0.0-90 wt % nitric acid solution at reflux to destroy undesired linear nitramines to provide crude hexahydro-1,3,5-trinitro-1,3,5-triazine;
ci.) collecting the crude hexahydro-1,3,5-trinitro-1,3,5-triazine from step (ch) by filtration;
cj.) recycling the anhydrous (0.0-0.50 wt % acetic anhydride) spent acid through the pre-distillation evaporators to complete the process cycle and supply 1,3,5,7-Tetraazatricy clo[3.3.1.13·7]decane dissolution, heel formation and ketene furnace conversion to acetic anhydride;
ck.) washing the crude Hexahydro-1,3,5-trinitro-1,3,5-triazine solids with hot water; and
cl.) recrystallization of the Hexahydro-1,3,5-trinitro-1,3,5-triazine via conventional processes.

14. The process of claim 10, wherein a part of the 0.0-0.50 wt % acetic anhydride spent acid is recycled to the subsequent Hexahydro-1,3,5-trinitro-1,3,5-triazine nitration heel, part of the 0.0-0.50 wt % acetic anhydride spent acid is recycled to dissolve the 1,3,5,7-tetraazatricyclo[3.3.1.13·7]decane, part of the 0.0-0.50 wt % acetic anhydride spent acid is processed through the pre-distillation evaporators followed by conversion to acetic anhydride in the ketene furnace; and the ammonium nitrate content in the 1,3,5,7-tetraazatricyclo[3.3.1.13·7]decane feed stream and acetic acid heel accounted for, and adjustments made in the subsequent standard heel formation.

15. A direct recycle of spent acid process for producing Octahydro-1,3,5,7-tetranitro1,3,5,7-tetrazocine, by nitrolysis of 1,3,5,7-tetraazatricyclo[3.3.1.13·7]decane, which comprises:
da.) while maintaining a temperature near 44° C., introducing a stream of 1,3,5,7-tetraazatricyclo[3.3.1.13.7]decane in acetic acid, a stream of ammonium nitrate in nitric acid, and a stream of acetic anhydride concurrently in proportions greater than is necessary to form Octahydro-1,3,5,7-tetranitro-1,3,5,7-tetrazocine intermediate 1 (stage 1), into a heel containing a majority acetic acid with acetic anhydride present in the heel;
db.) while maintaining a temperature near 44° C., introducing stream of ammonium nitrate in nitric acid and a stream of acetic anhydride concurrently in proportions greater than is necessary to a reaction mixture containing Octahydro-1,3,5,7-tetranitro-1,3,5,7-tetrazocine intermediate 1, to form Octahydro-1,3,5,7-tetranitro-1,3,5,7-tetrazocine intermediate 2 (stage 2);
dc.) while maintaining a temperature near 44° C., introducing stream of ammonium nitrate in nitric acid and a stream of acetic anhydride concurrently in proportions greater than is necessary to a reaction mixture containing Octahydro-1,3,5,7-tetranitro-1,3,5,7-tetrazocine intermediate 2, to form Octahydro-1,3,5,7-tetranitro-1,3,5,7-tetrazocine (stage 3);
dd.) heating the resulting slurry to 44° C. to affect nitrolysis of the Octahydro-1,3,5,7-tetranitro-1,3,5,7-tetrazocine intermediate to produce Octahydro-1,3,5,7-tetranitro1,3,5,7-tetrazocine and a crude reaction slurry containing acetic acid, acetic anhydride, nitric acid, ammonium nitrate;
de.) adding water to said slurry to consume the acetic anhydride and adjust the water content to approximately 0.0-20.0 wt % aqueous acetic acid;
df.) cooling the slurry to approximately 45° C.;
dg.) adding acetic anhydride to the said slurry to consume the water and adjust the acetic anhydride content to 0.0-0.50 wt % acetic anhydride;
dh.) adding ammonia (ammonium acetate) to the anhydrous, crude reaction slurry sufficient to neutralize the nitric acid;
di.) heating the neutralized slurry to 50-90° C. (a temperature sufficient to extract the RDX content to <2.0% of the HMX content) and separating the Octahydro-1,3,5,7-tetranitro-1,3,5,7-tetrazocine and the ammonium nitrate from the spent acid;
dj.) cooling the anhydrous acetic acid filtrate from step (di) to 25° C. and filtering precipitated solids, recovering spent acid;
dk.) stirring the Octahydro-1,3,5,7-tetranitro-1,3,5,7-tetrazocine from step (di) in a 0.0-90 wt % nitric acid solution at reflux to destroy undesired linear nitramines to provide crude Octahydro-1,3,5,7-tetranitro-1,3,5,7-tetrazocine;
dl.) collecting the crude Octahydro-1,3,5,7-tetranitro-1,3,5,7-tetrazocine from step (dk) by filtration;
dm.) recycling the filtered, anhydrous (0.0-0.50 wt % acetic anhydride) spent acid through the pre-distillation evaporators to complete the process cycle and supply 1,3,5,7-tetraazatricyclo[3.3.1.1 3·7]decane dissolution, subsequent heel formation and ketene furnace conversion to acetic anhydride; and
dn.) washing the filter cake with hot water and separating the Octahydro-1,3,5,7-tetranitro-1,3,5,7-tetrazocine from the hot water ammonium nitrate solution.

16. A direct recycle of spent acid process for producing Octahydro-1,3,5,7-tetranitro1,3,5,7-tetrazocine, by nitrolysis of 1,3,5,7-tetraazatricyclo[3.3.1.13·7]decane, which comprises:
ea.) while maintaining a temperature near 44° C., introducing a stream of 1,3,5,7-tetraazatricyclo[3.3.1.13.7]decane in acetic acid, a stream of ammonium nitrate in nitric acid, and a stream of acetic anhydride concurrently in proportions greater than is necessary to form Octahydro-1,3,5,7-tetranitro-1,3,5,7-tetrazocine intermediate 1 (stage 1), into a heel containing a majority acetic acid with acetic anhydride present in the heel;
eb.) while maintaining a temperature near 44° C., introducing stream of ammonium nitrate in nitric acid and a stream of acetic anhydride concurrently in proportions greater than is necessary to a reaction mixture containing Octahydro-1,3,5,7-tetranitro-1,3,5,7-tetrazocine intermediate 1, to form Octahydro-1,3,5,7-tetranitro-1,3,5,7-tetrazocine intermediate 2 (stage 2);
ec.) while maintaining a temperature near 44° C., introducing stream of ammonium nitrate in nitric acid and a stream of acetic anhydride concurrently in proportions greater than is necessary to a reaction mixture containing Octahydro-1,3,5,7-tetranitro-1,3,5,7-tetrazocine intermediate 2, to form Octahydro-1,3,5,7-tetranitro-1,3,5,7-tetrazocine (stage 3);
ed.) heating the resulting slurry to 44° C. to affect nitrolysis of the Octahydro-1,3,5,7-tetranitro-1,3,5,7-tetrazocine intermediate to produce Octahydro-1,3,5,7-tetranitrol3,5,7-tetrazocine and a crude reaction slurry containing acetic acid, acetic anhydride, nitric acid, ammonium nitrate;
ee.) adding water to said slurry to consume the acetic anhydride and adjust the water content to approximately 0.0-20.0 wt % aqueous acetic acid,
ef.) cooling the slurry to approximately 45° C.;
eg) adding acetic anhydride to the said slurry to consume the water and adjust the acetic anhydride content to 0.0-0.50 wt % acetic anhydride;
eh.) adding ammonia (ammonium acetate) to the anhydrous, crude reaction slurry sufficient to neutralize the nitric acid;
ei.) heating the neutralized slurry to 50-90° C. (a temperature sufficient to extract the RDX content to <2.0% of the HMX content) and separating the Octahydro-1,3,5,7-tetranitro-1,3,5,7-tetrazocine and the ammonium nitrate from the spent acid;
ej.) cooling the anhydrous acetic acid filtrate from step (ei) to 25° C. and filtering precipitated solids, recovering spent acid;
ek.) stirring the Octahydro-1,3,5,7-tetranitro-1,3,5,7-tetrazocine from step (ei) in a 0.0-90 wt % nitric acid solution at reflux to destroy undesired linear nitramines to provide crude hexahydro-1,3,5-trinitro-1,3,5-triazine;
el.) collecting the crude hexahydro-1,3,5-trinitro-1,3,5-triazine from step (ek) by filtration;
em.) recycling the filtered, anhydrous (0.0-0.50 wt % acetic anhydride) spent acid through the pre-distillation evaporators to complete the process cycle and supply 1,3,5,7-tetraazatricyclo[3.3.1.1 3·7]decane dissolution, subsequent heel formation and ketene furnace conversion to acetic anhydride; and
en.) washing the filter cake with hot water and separating the Octahydro-1,3,5,7-tetranitro-1,3,5,7-tetrazocine from the hot water ammonium nitrate solution.)

17. The process of claim 15, wherein a part of the 0.0-0.50 wt % acetic anhydride spent acid is from step (dj or ej) recycled to the subsequent Octahydro-1,3,5,7-tetranitro-1,3,5,7-tetrazocine heel, part of the 0.0-0.50 wt % acetic anhydride spent acid is recycled to dissolve the 1,3,5,7-tetraazatricyclo[3.3.1.13·7]decane, part of the 0.0-0.50 wt % acetic anhydride spent acid is processed through the pre-distillation evaporators followed by conversion to acetic anhydride in the ketene furnace; and the ammonium nitrate content is accounted for in the 1,3,5,7-tetraazatricyclo[3.3.1.13·7]decane acetic acid feed stream (x moles), and the subsequent nitrolysis heel. 1.0x moles nitric acid and 0.50x-1.0x moles 1,3,5,7-tetraazatricyclo[3.3.1.13·7]decane added to subsequent Octahydro-1,3,5,7-tetranitro-1,3,5,7-tetrazocine nitration heel.

18. The process of claim 15, wherein a part of the 0.0-0.50 wt % acetic anhydride spent acid is recycled to the subsequent Hexahydro-1,3,5-trinitro-1,3,5-triazine nitration heel, part of the 0.0-0.50 wt % acetic anhydride spent acid is recycled to dissolve the 1,3,5,7-tetraazatricyclo[3.3.1.13·7]decane (to be employed in the Hexahydro-1,3,5-trinitro-1,3,5-triazine process), part of the 0.0-0.50 wt % acetic anhydride spent acid is processed through the pre-distillation evaporators followed by conversion to acetic anhydride in the ketene furnace; and the ammonium nitrate content in the 1,3,5,7-tetraazatricyclo[3.3.1.13·7]decane feed stream and acetic acid heel accounted for, and adjustments made in the standard heel formation.
